(19) United States
(12) Patent Application Publication
(10) Pub. No.: US 2006/0287582 A1
(43) Pub. Date: Dec. 21, 2006

(54) Endoscopic system

(75) Inventor: Masato Toda, Hachioji-shi (JP)

Correspondence Address: OSTROLENK FABER GERB & SOFFEN, 1180 AVENUE OF THE AMERICAS, NEW YORK, NY, 100368403, US

(73) Assignee: Olympus Medical Systems Corporation

(21) Appl. No.: 11/453,372

(22) Filed: Jun. 15, 2006

(30) Foreign Application Priority Data
Jun. 16, 2005 (JP) 2005-176551

(51) Int. Cl. A61B 1/06

(52) U.S. Cl. 600/178; 600/182; 600/179

(57) ABSTRACT

An endoscopic system includes an endoscope and an illuminating device. The endoscope includes an elongated insertion section having a distal end and a proximal end, an operation section disposed in the proximal end of the insertion section, and a light guide inserted through the operation section and the insertion section, having an incidence end face in the operation section, and having an emission end face in the distal end of the insertion section. The illuminating device is optically connected to the incidence end face of the light guide disposed in the operation section, and has a light source which feeds light to the incidence end face. The light guide is provided with a light emitting element which emits light in parallel with the light-source light emitted from the emission end face.

### CROSS-REFERENCE TO RELATED APPLICATIONS

This application is based upon and claims the benefit of priority from prior Japanese Patent Application No. 2005-176551, filed Jun. 16, 2005, the entire contents of which are incorporated herein by reference.

### BACKGROUND OF THE INVENTION

1. Field of the Invention

The present invention relates to an endoscopic system which is capable of usually observing a subject and which is especially capable of observing a subject by use of special light.

2. Description of the Related Art

In, for example, Jpn. Pat. Appln. KOKAI Publication No. 2002-336196, there is disclosed an endoscopic system in which fluorescence observation is possible. In this endoscopic system, a subject is irradiated with light (e.g., fluorescence excitation light) having a specific wavelength, and light reflected by the subject or self-fluorescence is observed. This endoscopic system switchably includes an RGB filter and a three-color filter for special-light observation in an optical path of usual white light. To observe the self-fluorescence, the system is used in a state in which the special-light observing three-color filter is disposed along the optical path of the usual white light. In this case, the subject is irradiated with, for example, the fluorescence excitation light, the self-fluorescence emitted from the subject is picked up by an image sensor, and fluorescence observation can be performed.

In, for example, Jpn. Pat. Appln. KOKAI Publication No. 2003-190091, there is disclosed a fluorescence observing illuminative probe for use in the endoscopic system in which the fluorescence observation is possible. The probe includes a light emitting element which emits special light on a distal end of the probe. When the probe is inserted through a treatment instrument insertion channel of an endoscope, the subject can be illuminated with light emitted from the light emitting element of the probe.

### BRIEF SUMMARY OF THE INVENTION

According to an aspect of the present invention, there is provided an endoscopic system including an endoscope and an illuminating device. The endoscope includes an elongated insertion section having a distal end and a proximal end; an operation section disposed in the proximal end of the insertion section; and a light guide inserted through the operation section and the insertion section, having an incidence end face in the operation section, and having an emission end face in the distal end of the insertion section. The illuminating device is optically connected to the incidence end face of the light guide disposed in the operation section, and has a light source which feeds light to the incidence end face. The light guide is provided with a light emitting element which emits light in parallel with the light-source light emitted from the emission end face.

According to the other aspect of the present invention, there is provided an endoscopic system including an endoscope and an illuminating device. The endoscope includes an elongated insertion section having a distal end and a proximal end; an operation section disposed in the proximal end of the insertion section; and a light guide inserted through the operation section and the insertion section, having an incidence end face in the operation section, and having an emission end face in the distal end of the insertion section. The illuminating device is optically connected to the incidence end face of the light guide disposed in the operation section, and has a light source which feeds light to the incidence end face. The insertion section is provided with, on the distal end of the insertion section, a light emitting element which emits light in parallel with the light-source light emitted from the emission end face.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic perspective view showing a component of an endoscopic system in a first embodiment of the present invention;

FIG. 2A is a schematic diagram showing a distal-end face of an insertion section an endoscope of an endoscopic system in the first embodiment;

FIG. 2B is a schematic sectional view cut along the 2B-2B line of FIG. 2A, especially showing an observation optical system and an illumination optical system disposed in the endoscope of the endoscopic system in the first embodiment;

FIG. 3A is a schematic diagram showing a distal-end face of an insertion section of an endoscope of an endoscopic system in a second embodiment;

FIG. 3B is a schematic sectional view cut along the 3B-3B line of FIG. 3A, especially showing an observation optical system and an illumination optical system disposed in the endoscope of the endoscopic system in the second embodiment;

FIG. 4A is a schematic diagram showing a distal-end face of an insertion section of an endoscope of an endoscopic system in a third embodiment;

FIG. 4B is a schematic sectional view cut along the 4B-4B line of FIG. 4A, especially showing an observation optical system and an illumination optical system disposed in the endoscope of the endoscopic system in the third embodiment;

FIG. 5A is a schematic diagram showing a distal-end face of an insertion section of an endoscope of an endoscopic system in a fourth embodiment;

FIG. 5B is a schematic sectional view cut along the 5B-5B line of FIG. 5A, especially showing an observation optical system and an illumination optical system disposed in the endoscope of the endoscopic system in the fourth embodiment;

FIG. 6A is a schematic diagram showing a distal-end face of an insertion section of an endoscope of an endoscopic system in a fifth embodiment;

FIG. 6B is a schematic sectional view cut along the 6B-6B line of FIG. 6A, especially showing an observation optical system and an illumination optical system disposed in the endoscope of the endoscopic system in the fifth embodiment; and

FIG. 7 is a schematic diagram showing an RGB filter which can be disposed in a light source device of the endoscopic system in the first to fifth embodiments.

### DETAILED DESCRIPTION OF THE INVENTION

There will be described hereinafter the best mode for carrying out this invention with reference to the drawings.

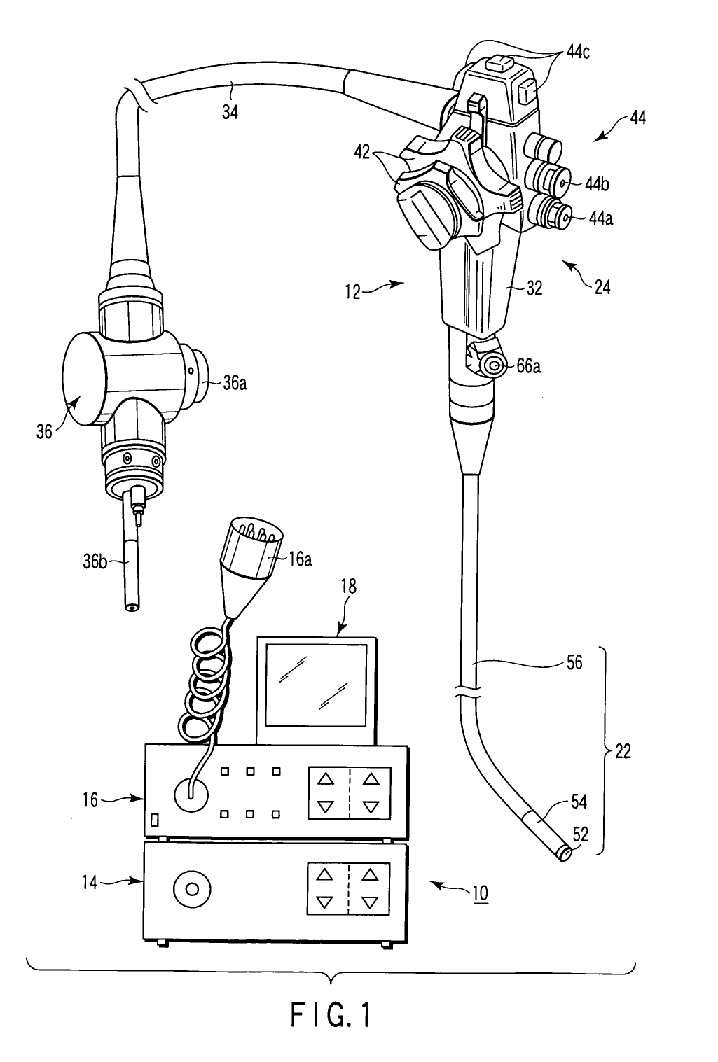

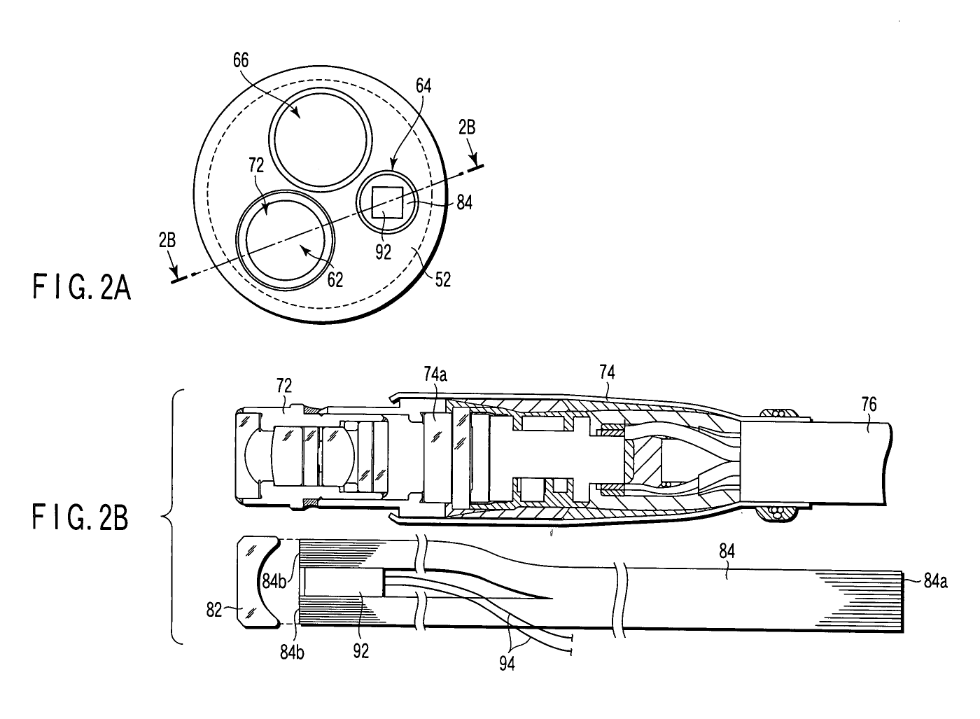

A first embodiment will be described with reference to FIGS. 1 to 2B.

As shown in FIG. 1, an endoscopic system 10 of the embodiment includes an endoscope 12, a light source device 14, a video processor 16 and a monitor 18.

The light source device 14 is detachably connected to the endoscope 12, and supplies illuminative light to the endoscope 12. The light source device 14 includes, for example, a xenon lamp (not shown) as an illuminative light source (white light). The video processor 16 is detachably connected to the endoscope 12, and controls the endoscope 12, or processes a signal transmitted from the endoscope 12. The monitor 18 is detachably connected to the video processor 16, and displays a video signal output from the video processor 16.

The endoscope 12 includes an elongated insertion section 22 and an operation section 24 disposed in a proximal end of the insertion section 22.

The operation section 24 includes an operation section main body 32 and a universal cord 34. The universal cord 34 extends from the operation section main body 32, and includes a connector 36 detachably connected to the light source device 14 or the video processor 16. The connector 36 includes an electric connector 36a and a light guide connector 36b.

The light guide connector 36b is detachably connected to the light source device 14. When the light guide connector 36b is connected to the light source device 14, the light guide connector 36b is optically connected to the above light source of the light source device 14. The electric connector 36a is detachably connected to an extension connector 16a of the video processor 16. When the electric connector 36a is connected to the video processor 16, the electric connector 36a is electrically connected to the video processor 16. That is, an electric portion of the endoscope 12 is controlled by the video processor 16.

In the operation section main body 32, there are disposed bending operation knobs 42 which bend a bent portion 54 of the insertion section 22 described later. The operation section main body 32 is further provided with a switch portion 44 having a suction switch 44a and an air-and-water feeding switch 44b operated to suck a living tissue or the like disposed in the vicinity of the distal end of the insertion section 22. The switch portion 44 having the suction switch 44a and the air-and-water feeding switch 44b is manually operated, when the operation section main body 32 is hand-held by an operator.

It is to be noted that the switch portion 44 further includes observation mode changeover switches 44c. The observation mode changeover switches 44c can be switched to a usual mode in which usual observation is performed, a special observation mode in which special observation is performed using special light, and a mixed observation mode in which a light source in performing the usual observation is mixed with a light source in performing the special observation. Since an electric portion of the endoscope 12 is electrically connected to the video processor 16 as described above, a switched state of the switch 44 is controlled by the video processor 16.

The insertion section 22 includes a distal-end hard portion 52, the bent portion 54 and a flexible tube 56 from a distal end toward a proximal end of the insertion section.

The flexible tube 56 has an appropriate flexibility, and extends from the operation section main body 32. The bent portion 54 is connected to the bending operation knob 42 of the operation section 24 by a wire (not shown) inserted through the flexible tube 56. Therefore, the bent portion 54 is bent in a desired direction by an operation of the bending operation knob 42 of the operation section 24.

As shown in FIG. 2A, in the distal-end hard portion 52 of the insertion section 22, there are arranged an observation optical system 62, an illumination optical system 64 and a treatment instrument insertion channel 66. The observation optical system 62, the illumination optical system 64 and the treatment instrument insertion channel 66 are arranged in the operation section 24 through the bent portion 54 and the flexible tube 56 of the insertion section 22. Among them, the observation optical system 62 and the illumination optical system 64 are inserted from the distal end of the insertion section 22 through the operation section main body 32 into the connector 36 which is the end portion of the universal cord 34. As shown in FIG. 1, a forceps valve 66a is disposed in the proximal end of the treatment instrument insertion channel 66, that is, a position where forceps or the like are to be inserted.

As shown in FIG. 2B, the observation optical system 62 includes an objective lens unit 72, an image pickup unit 74 disposed in a proximal end of the objective lens unit 72, and a signal cable 76 extended from a proximal end of the image pickup unit 74.

The image pickup unit 74 includes an image sensor 74a. The image sensor 74a has a characteristic that an image observed with special light can be picked up at a time when a subject is illuminated with the special light, in addition to a characteristic that an image observed with usual light can be picked up. That is, as the image sensor 74a, an element is used in which sensitivity is appropriately set so as to take in not only a wavelength of the usual light but also a wavelength of the special light. Therefore, the image sensor 74a having a broad band is used. The image sensor 74a preferably has, for example, an electronic filter which is capable of picking up the image while emphasizing an appropriate wavelength, that is, which cuts light having an appropriate wavelength.

To perform such control, the image sensor 74a is used in a state in which the sensor is electrically connected to the video processor 16. A plurality of signal lines are extended from the image sensor 74a. These signal lines are put together in the signal cable 76. The signal cable 76 is electrically connected to the electric connector 36a.

The objective lens unit 72 includes a lens system in which a plurality of lenses are combined. These lenses are arranged movably along an axial direction. Therefore, the image sensor 74a can pick up a subject image in a state in which the subject image is focused on the image sensor 74a.

On the other hand, as shown in FIG. 2B, the illumination optical system 64 includes an illumination lens 82 and a light guide 84 disposed in a proximal end of the illumination lens 82. An incidence end face 84a of the light guide 84 is disposed in the light guide connector 36b, and an emission end face 84b of the light guide is disposed in a distal end of the insertion section 22. That is, the light guide 84 is inserted through substantially the whole insertion section 22 and operation section 24.

The light guide 84 is bunched by a plurality of fibers aligned in one direction. The fibers are formed so that a sectional shape of the light guide 84 is circular. Each of the fibers is made of, for example, multicomponent-based glass, quartz or the like. The light guide 84 formed in this manner can transmit (guide) usual light for usual observation or light having a wavelength for special-light observation with little transmission loss.

The illumination lens 82 is disposed in a position which faces the emission end face 84b of the light guide 84. That is, the illumination lens 82 is disposed in the distal end of the insertion section 22. On the other hand, the light source of the light source device 14 is disposed in a position which faces the incidence end face 84a of the light guide 84. It is to be noted that a condensing lens (not shown) is disposed between the light source and the incidence end face 84a of the light guide 84. Therefore, light emitted from the light source is condensed on the incidence end face 84a while striking on the face. The light, which has struck on the incidence end face 84a of the light guide 84, is emitted from the emission end face 84b through the light guide 84, and emitted from the illumination lens 82 to the outside of the endoscope 12.

In this embodiment, in the distal end of the light guide 84, a light emitting element 92 is buried such as a light emitting diode (LED) or a laser diode (LD). Lead wires 94 are extended from this light emitting element 92. These lead wires 94 are electrically connected to the electric connector 36a through the insertion section 22 and the operation section 24.

It is to be noted that the light emitting element 92 has a characteristic that ultraviolet light having a wavelength of, for example, about 350 nm to 400 nm be emitted. The light emitting element 92 further has a characteristic that infrared light having a wavelength, for example, about 700 nm to 750 nm be emitted. The light source has at least a characteristic that visible light having a wavelength of about 400 nm to 700 nm be emitted.

There will be described a function of the endoscopic system 10 in this embodiment. The endoscopic system 10 of this embodiment has, for example, three observation modes. A first observation mode is a usual-light observation mode for use in a state in which only white light is emitted. A second observation mode is a special-light observation mode for use in a state in which the light is emitted from the only light emitting element 92. A third observation mode is a mixed observation mode for use in a state in which usual light is combined with special light.

It is to be noted that the light emitting element 92 for use in the special-light observation mode is sometimes different from that for use in the mixed observation mode. That is, the element does not have to be the same, but in the following description, it is assumed that the elements are the same.

First, the usual-light observation mode will be described, and then the special-light observation mode and the mixed observation mode will be described.

In the usual-light observation mode, the insertion section 22 of the endoscope 12 is inserted into an appropriate position. That is, the endoscope 12 is operated in the same manner as in a usual operation, and the distal end of the insertion section 22 is introduced into a position of a subject to be observed.

In this case, the white light is emitted from the light source device 14 to strike on the light guide connector 36b of the endoscope 12. Therefore, the white light is guided from the incidence end face 84a to the emission end face 84b of the light guide 84. The light emitted from the emission end face 84b of the light guide 84 illuminates the subject through the illumination lens 82.

The image of the illuminated subject is picked up by the image pickup unit 74 through the objective lens unit 72, and a signal of the image is successively input into the video processor 16 through the signal cable 76. Therefore, the processor 16 usually processes the image to display the subject image in the monitor 18.

Next, there will be described a case where the same subject is observed in the special-light observation mode. In this case, the observation mode changeover switches 44c of the switch portion 44 of the operation section 24 is operated to switch the observation mode from the usual-light observation mode to the special-light observation mode. In this case, an operation signal of the observation mode changeover switch 44c is transmitted from the switch 44c to the processor 16.

The processor 16 turns off the white light, and turns on the light emitting element 92. The illuminative light emitted from the light emitting element 92 illuminates the subject through the illumination lens 82. In this case, since the light is not passed through the light guide 84, the subject is efficiently illuminated.

The image of the illuminated subject is picked up by the image pickup unit 74 through the objective lens unit 72, and the signal of the image is input into the processor 16 through the signal cable 76. Therefore, the processor 16 processes the special-light image based on a type of the light emitting element 92 to display the subject image in the monitor 18.

Next, there will be described a case where the same subject is observed in the mixed observation mode. In this case, the observation mode changeover switches 44c of the switch portion 44 of the operation section 24 is operated to switch the observation mode to the mixed observation mode. In this case, the operation signal of the observation mode changeover switch 44c is transmitted from the switch 44c to the processor 16.

In a case where the special-light observation mode is switched to the mixed observation mode, the processor 16 further turns on the light source of the white light. In a case where the usual-light observation mode is switched to the mixed observation mode, the light emitting element 92 is further turned on. That is, both of the light emitting element 92 and the white light are turned on.

Therefore, the white light and the light from the light emitting element 92 illuminates the subject through the illumination lens 82. The image of the illuminated subject is picked up by the image pickup unit 74 through the objective lens unit 72, and the signal of the image is input into the processor 16 through the signal cable 76. Therefore, the processor 16 performs image processing for obtaining a desired observation image, such as emphasizing of the light having the appropriate wavelength, and displays the subject image in the monitor 18.

As described above, according to this embodiment, the following can be said.

In a case where the subject is observed in the special-light observation mode, the optical filter for illuminating the subject with the special light does not have to be disposed in the light source device 14. Therefore, the light source device 14 can be miniaturized and lightened. Therefore, costs of the endoscopic system 10 can be reduced as a whole.

Moreover, when the light emitting element 92 is simply used as such, it is possible to emit the light having the wavelength. This light has been difficult to emit in a state in which the optical filter is disposed along the optical path of the white light. That is, when the characteristic of the light emitting element 92 is selected, various special-light observations can be performed.

Furthermore, a position where the light is emitted from the light guide 84 is the same as a position where the light is emitted from the light emitting element 92. Therefore, in a case where the same position of the subject is irradiated with light having different characteristics, when the observation mode is switched, the subject can be compared and considered.

In addition, since the treatment instrument insertion channel 66 can be used for other forceps (not shown), treatment using the forceps or the like can easily be performed.

It is to be noted that in a case where the special-light observation mode is utilized, as the light emitting element 92, various types of special light sources can be used such as a source which emits the ultraviolet light and the infrared light and a source which emits the fluorescence excitation light to excite fluorescence.

Moreover, it has been described in this embodiment that the lead wires 94 of the light emitting element 92 are connected to the electric connector 36a, but a power supply for the light emitting element is disposed in the light source device 14, and the lead wires 94 may be connected to the power supply for the light emitting element.

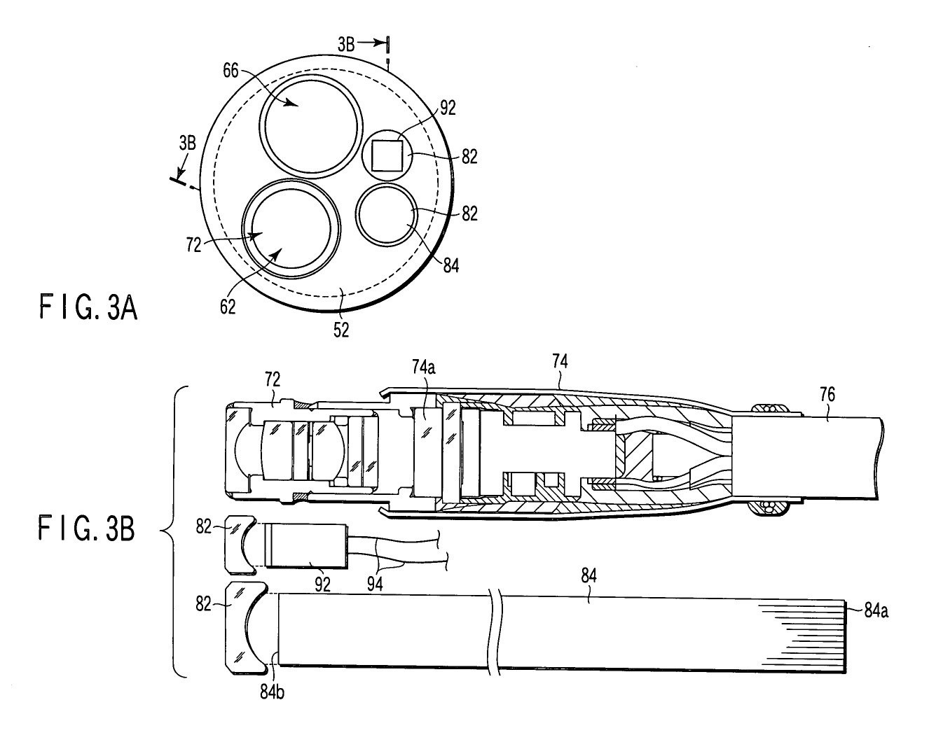

Next, there will be described a second embodiment with reference to FIGS. 3A and 3B. This embodiment is a modification of the first embodiment, the same members as those described in the first embodiment are denoted with the same reference numerals, and detailed description thereof is omitted.

As shown in FIG. 3B, a light emitting element 92 is removed from an emission end face 84b of a light guide 84 in this embodiment, and the emission end face 84b is integrated into one face. Instead, as shown in FIGS. 3A and 3B, the light emitting element 92 and the emission end face 84b of the light guide 84 are separately arranged. An illumination lens 82 is disposed in a light emitting position of this light emitting element 92.

Since a function and an effect of an endoscopic system 10 of the present embodiment are the same as those of the first embodiment, description thereof is omitted.

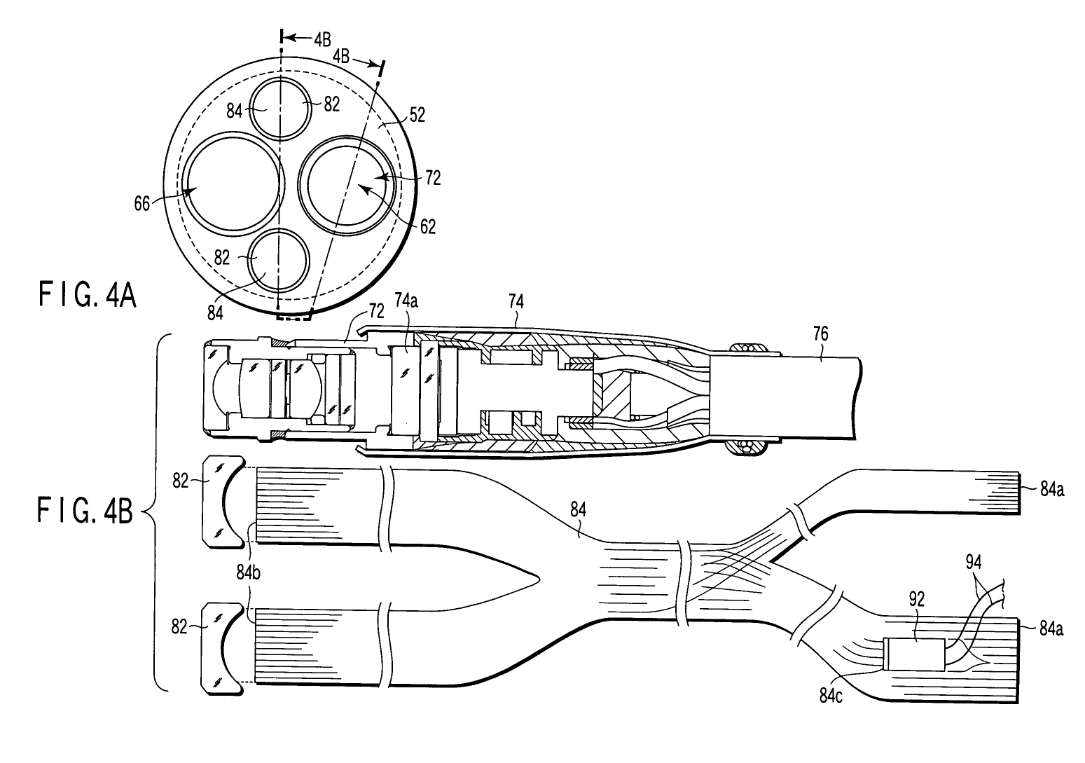

Next, there will be described a third embodiment with reference to FIGS. 4A and 4B. This embodiment is a modification of the first embodiment, the same members as those described in the first embodiment are denoted with the same reference numerals, and detailed description thereof is omitted.

As shown in FIG. 4B, in the present embodiment, a light emitting element 92 (see FIG. 2B) is removed from an emission end face 84b of a light guide 84, and the emission end face is branched into two faces. That is, the light guide 84 includes two emission end faces 84b and two incidence end faces 84a. Among them, two emission end faces emit, with a substantially equal ratio, the light which has entered two incidence end faces 84a.

In positions which face two emission end faces 84b, illumination lenses 82 are disposed, respectively.

There are formed two incidence end faces 84a of the light guide 84. That is, the light guide 84 is branched into two in an appropriate position between the emission end face 84b and the incidence end face 84a. The light emitting element 92 is buried in the vicinity of one of two incidence end faces 84a. Lead wires 94 extended from this light emitting element 92 are electrically connected to, for example, an electric connector 36a.

The incidence end face 84a of the light guide 84 in which the light emitting element 92 is buried in this manner is disposed in a position which faces a light source (not shown). The other incidence end face 84a in which any light emitting element 92 is not buried is similarly disposed in a position which faces the same light source.

It is to be noted that the light guide 84 is formed in a randomly mixed state between the incidence end face 84a and the emission end face 84b so that the light which has entered two incidence end faces 84a is substantially uniformly emitted from the emission end faces 84b. That is, fibers arranged in two incidence end faces 84a, respectively, are mixed in the emission end faces 84b. Therefore, when the light enters two incidence end faces 84a, the light is emitted at an equal ratio from two emission end faces 84b, respectively.

It is to be noted that in the present embodiment, two emission end faces 84b are shown in FIG. 4B, but it is preferable that the emission end faces 84b are integrated into one face.

Next, there will be described a function of an endoscopic system 10 of the present embodiment.

In a usual-light observation mode, an insertion section 22 of an endoscope 12 is inserted into an appropriate position. That is, the endoscope 12 is operated as usual, and a distal end of the insertion section 22 is introduced into a position of a subject to be observed.

In this case, only white light is emitted from a light source device 14 into a light guide connector 36b of the endoscope 12. Therefore, the white light is guided from two incidence end faces 84a facing the light source of the light guide 84 to the emission end faces 84b, respectively. The light emitted from two emission end faces 84b of the light guide 84 illuminates the subject through the illumination lenses 82. In this case, the light emitted from the emission end faces 84b of the light guide 84 is uniformly emitted without deviating.

An image of the illuminated subject is picked up by an image pickup unit 74 through an objective lens unit 72, and a signal of the image is successively input into a processor 16 through a signal cable 76. Therefore, the processor 16 usually processes the image to display the subject image in a monitor 18.

Next, there will be described a case where the same subject is observed in a special-light observation mode. In this case, an observation mode changeover switches 44c of a switch portion 44 of an operation section 24 is operated to switch the observation mode from the usual-light observation mode to the special-light observation mode. In this case, an operation signal of the observation mode changeover switch 44c is transmitted from the switch 44c to the processor 16.

The processor 16 turns off the white light, and turns on the light emitting element 92. As to illuminative light emitted from the light emitting element 92, special light from the light emitting element 92 is guided to the emission end faces 84b of the light guide 84 from the incidence end faces 84a facing an emission end face of the light emitting element 92. The light emitted from the emission end faces 84b of the light guide 84 illuminates the subject through the illumination lenses 82. In this case, the light emitted from the emission end faces 84b of the light guide 84 is uniformly emitted without deviating.

The image of the illuminated subject is picked up by the image pickup unit 74 through the objective lens unit 72, and the signal of the image is input into the processor 16 through the signal cable 76. Therefore, the processor 16 processes the special-light image based on a type of the light emitting element 92 to display the subject image in the monitor 18.

Next, there will be described a case where the same subject is observed in a mixed observation mode. In this case, the observation mode changeover switches 44c of the switch portion 44 of the operation section 24 is operated to switch the observation mode to the mixed observation mode. In this case, the operation signal of the observation mode changeover switch 44c is transmitted from the switch 44c to the processor 16. In this case, both of the light emitting element 92 and the white light are turned on.

Therefore, both of the white light and the light from the light emitting element 92 illuminate the subject through the light guide 84 and the illumination lenses 82. The image of the illuminated subject is picked up by the image pickup unit 74 through the objective lens unit 72, and the signal of the image is input into the processor 16 through the signal cable 76. Therefore, the processor 16 performs image processing for obtaining a desired observation image, such as emphasizing of the light having the appropriate wavelength, and displays the subject image in the monitor 18.

As described above, according to this embodiment, the following can be said.

Since the light emitting element 92 does not have to be disposed in the distal end of the insertion section 22, special observation can be performed using the light emitting element 92 without enlarging an outer diameter of the distal end of the insertion section 22. The light from the light emitting element 92 and the light from the light source are emitted from the same position. Therefore, when the observation mode is switched, the same position can be observed in comparison with that of another observation mode.

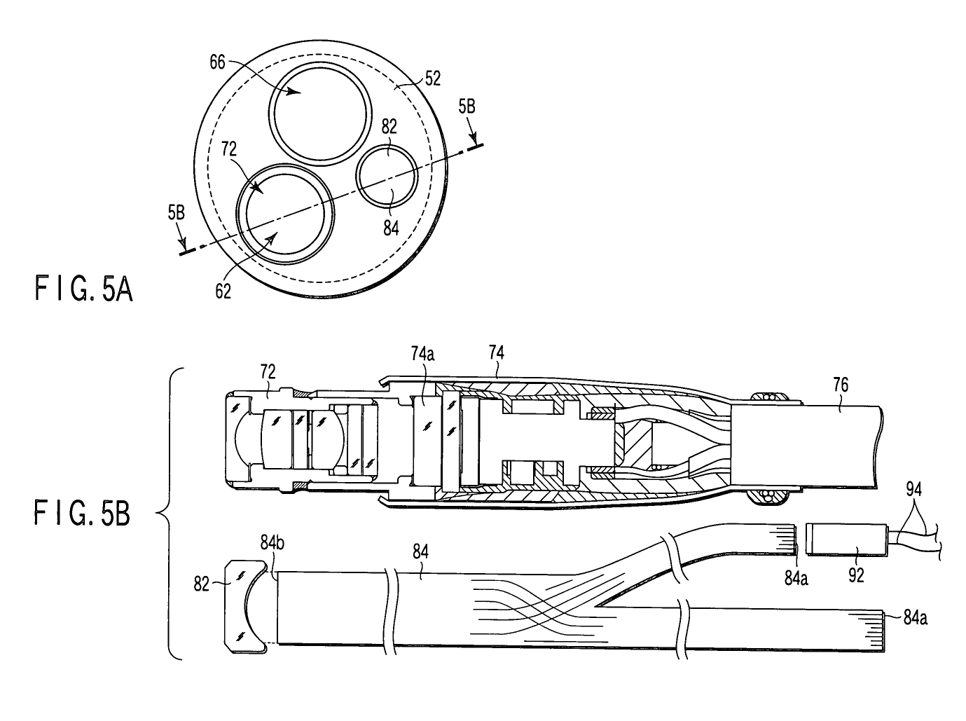

Next, there will be described a fourth embodiment with reference to FIGS. 5A and 5B. This embodiment is a modification of the first embodiment, the same members as those described in the first embodiment are denoted with the same reference numerals, and detailed description thereof is omitted.

As shown in FIG. 5B, in the present embodiment, a light emitting element 92 is removed from an emission end face 84b of a light guide 84, and the emission end faces 84b are integrated into one face. On the other hand, two incidence end faces 84a are formed. That is, the light guide 84 is branched into two in an appropriate position between the emission end face 84b and the incidence end face 84a.

One of two incidence end faces 84a is disposed in a position which faces a light source (not shown). On the other hand, the other incidence end face 84a is disposed in a position which faces the light emitting element 92.

It is to be noted that the light guide 84 is formed in a randomly mixed state between the incidence end face 84a and the emission end face 84b so that the light which has entered two incidence end faces 84a is substantially uniformly emitted from the emission end face 84b. That is, fibers arranged in two incidence end faces 84a, respectively, are mixed in the emission end face 84b. Therefore, the light which has entered two incidence end faces 84a is substantially uniformly emitted.

Moreover, the light emitting element 92 is changeably disposed in, for example, a light source device 14. That is, the light emitting element 92 of, for example, a fitted type is disposed in the light source device 14. Therefore, when the light emitting element 92 is changed, various types of special light can selectively be utilized in the endoscopic system 10. Therefore, various observation modes can selectively be utilized.

Next, there will be described a function of the endoscopic system 10 of the present embodiment.

In a usual-light observation mode, an insertion section 22 of an endoscope 12 is inserted into an appropriate position. That is, the endoscope 12 is operated as usual, and a distal end of the insertion section 22 is introduced beforehand in a position of a subject to be observed.

In this case, only white light is emitted from the light source device 14 into a light guide connector 36b of the endoscope 12. Therefore, the white light is guided from the incidence end face 84a facing the light source of the light guide 84 to the emission end face 84b. The light emitted from the emission end face 84b of the light guide 84 illuminates the subject through an illumination lens 82. In this case, the light emitted from the emission end face 84b of the light guide 84 is uniformly emitted without deviating.

An image of the illuminated subject is picked up by an image pickup unit 74 through an objective lens unit 72, and a signal of the image is successively input into a processor 16 through a signal cable 76. Therefore, the processor 16 usually processes the image to display the subject image in a monitor 18.

Next, there will be described a case where the same subject is observed in a special-light observation mode. In this case, an observation mode changeover switches 44c of a switch portion 44 of an operation section 24 is operated to switch the observation mode from the usual-light observation mode to the special-light observation mode. In this case, an operation signal of the observation mode changeover switch 44c is transmitted from the switch 44c to the processor 16.

The processor 16 turns off the white light, and turns on the light emitting element 92. As to illuminative light emitted from this light emitting element 92, special light from the light emitting element 92 is guided to the emission end face 84b of the light guide 84 from the incidence end face 84a facing the light emitting element 92. The light emitted from the emission end face 84b of the light guide 84 illuminates the subject through the illumination lens 82. In this case, the light emitted from the emission end face 84b of the light guide 84 is uniformly emitted without deviating.

The image of the illuminated subject is picked up by the image pickup unit 74 through the objective lens unit 72, and the signal of the image is input into the processor 16 through the signal cable 76. Therefore, the processor 16 processes the special-light image based on a type of the light emitting element 92 to display the subject image in the monitor 18.

Next, there will be described a case where the same subject is observed in a mixed observation mode. In this case, the observation mode changeover switches 44c of the switch portion 44 of the operation section 24 is operated to switch the observation mode to the mixed observation mode. In this case, the operation signal of the observation mode changeover switch 44c is transmitted from the switch 44c to the processor 16. In this case, both of the light emitting element 92 and the white light are turned on.

Therefore, both of the white light and the light from the light emitting element 92 illuminate the subject through the light guide 84 and the illumination lens 82. The image of the illuminated subject is picked up by the image pickup unit 74 through the objective lens unit 72, and the signal of the image is input into the processor 16 through the signal cable 76. Therefore, the processor 16 performs image processing for obtaining a desired observation image, such as emphasizing of the light having the appropriate wavelength, and displays the subject image in the monitor 18.

As described above, according to this embodiment, the following can be said.

Since the light emitting element 92 does not have to be disposed in the distal end of the insertion section 22, special observation can be performed using the light emitting element 92 without enlarging an outer diameter of the distal end of the insertion section 22. The light from the light emitting element 92 and the light from the light source are emitted from the same position. Therefore, when the observation mode is switched, the same position can be observed in comparison with that of another observation mode.

In the present embodiment, the light emitting element 92 is fitted into the light source device 14. That is, it has been described that the light emitting element 92 is changeable, but it is preferable that the light emitting element 92 is fitted into a proximal end of the insertion section 22 or an operation section main body 32. In this case, at least one incidence end face 84a of the light guide 84 is disposed in the proximal end of the insertion section 22 or the operation section main body 32. That is, the light guide 84 is branched in the insertion section 22. In this case, it is possible to obtain a function and an effect which are similar to those obtained in a case where the light emitting element 92 is changeably fitted into the light source device 14. It is to be noted that in a case where the light emitting element is disposed in the proximal end of the insertion section 22, it is preferable that the incidence end faces 84a of the light guide 84 are disposed, for example, in the vicinity of a forceps stopper 66a of a treatment instrument insertion channel.

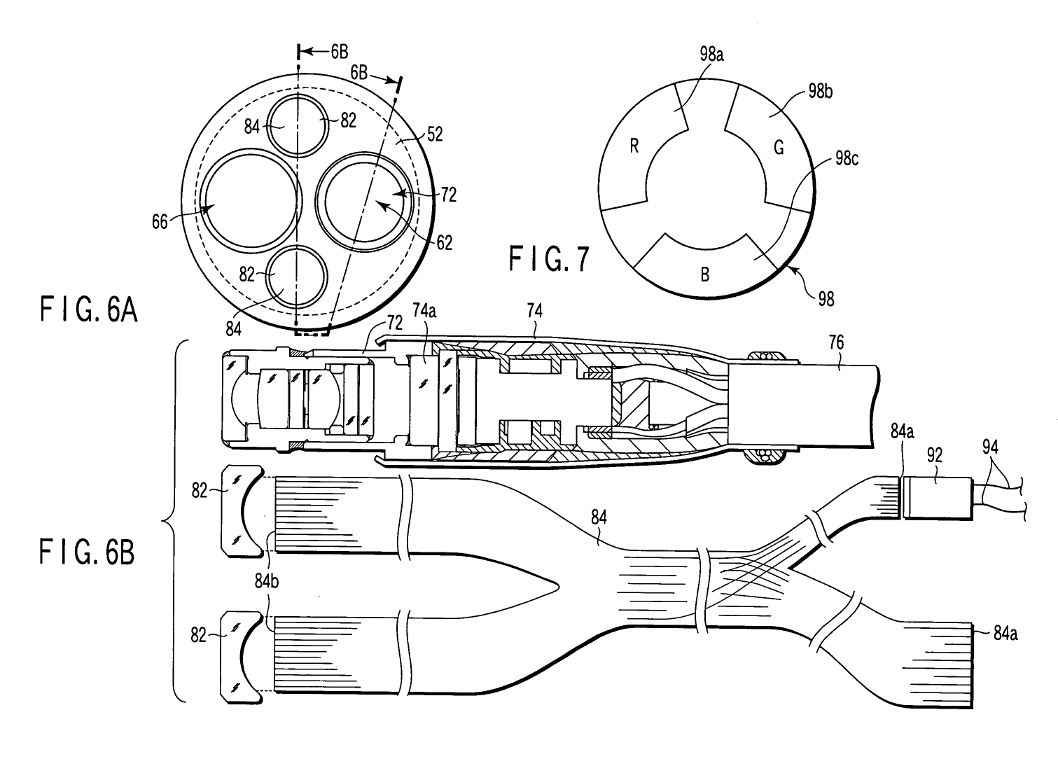

Next, there will be described a fifth embodiment with reference to FIGS. 6A and 6B. This embodiment is a modification of the fourth embodiment, the same members as those described in the fourth embodiment are denoted with the same reference numerals, and detailed description thereof is omitted.

In the present embodiment, an emission end face 84b of a light guide 84 is branched into two faces. That is, the light guide 84 includes two emission end face 84b and two incidence end faces 84a. Among them, two emission end faces emit, with a substantially equal ratio, light which has entered two incidence end faces 84a.

In positions which face two emission end faces 84b, illumination lenses 82 are disposed, respectively.

Since a function and an effect of an endoscopic system 10 of the present embodiment are the same as those of the fourth embodiment, description thereof is omitted.

It is to be noted that it has been described in the above first to fifth embodiments that the white light is used as the light source, but another light source may be used. It is also preferable that, for example, a disc-like RGB filter 98 shown in FIG. 7 is disposed between the light source and the incidence end face 84a of the light guide 84. That is, it is further preferable that the RGB filter 98 is disposed in the light source device 14. In this RGB filter 98, an R-filter 98a, a G-filter 98b and a B-filter 98c are circumferentially arranged, and rotated along the optical path from the light source when arranged in order. That is, the RGB filter 98 is disposed in a plane which crosses the optical path of the light source at right angles.

Therefore, when the R-filter 98a is disposed along the optical path, R-light enters the light guide 84. When the G-filter 98b is disposed along the optical path, G-light enters the light guide 84. When the B-filter 98c is disposed along the optical path, B-light enters the light guide 84. The processor 16 processes the subject image to obtain the image while judging the emitted light through the RGB filter 98. That is, after storing once in a memory (not shown) image data picked up at a time when the R-light is emitted, image data picked up at a time when G-light is emitted, and image data picked up at a time when B-light is emitted, the data is read together to constitute the subject image. In this case, a color image of the subject is displayed in the monitor 18.

In a case where the subject is observed in the mixed observation mode, it is preferable that the light emitting element 92 emits the light in a timing matched with an emission timing of the R-light, the G-light or the B-light. Such timing is controlled by the video processor 16. In this case, the R-light and the light from the light emitting element 92 can be emitted together from the emission end face 84b of the light guide 84 to pick up an image of reflected light (including, e.g., fluorescence) of the emitted light. This also applies to a case where the G-light or the B-light is emitted.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general invention concept as defined by the appended claims and their equivalents.

What is claimed is:

1. An endoscopic system comprising:
an endoscope including:
an elongated insertion section having a distal end and a proximal end;
an operation section disposed in the proximal end of the insertion section; and
a light guide inserted through the operation section and the insertion section, having an incidence end face in the operation section, and having an emission end face in the distal end of the insertion section; and
an illuminating device optically connected to the incidence end face of the light guide disposed in the operation section, and having a light source which feeds light to the incidence end face,
wherein the light guide is provided with a light emitting element which emits light in parallel with the light-source light emitted from the emission end face.

2. The endoscopic system according to claim 1, wherein the light guide further includes an incidence end face other than the incidence end face of the operation section, and
the light emitting element is disposed in a position which faces the other incidence end face.

3. The endoscopic system according to claim 2, wherein the other incidence end face is disposed in the proximal end of the insertion section, and
the light emitting element is detachably disposed in a position which faces the other incidence end face.

4. The endoscopic system according to claim 2, wherein the operation section includes an operation section main body disposed in the proximal end of the insertion section,
the other incidence end face is disposed in the operation section main body, and
the light emitting element is detachably disposed in a position which faces the other incidence end face.

5. The endoscopic system according to claim 2, wherein the light emitting element emits the light in a state in which the light is emitted from the light source of the illuminating device.

6. The endoscopic system according to claim 5, wherein an RGB filter which feeds R-light, G-light and B-light to the light guide in order by rotation is disposed between the light source and the incidence end face of the light guide, and
the light emitting element emits the light while matching with an incidence timing of the R-light, the G-light and the B-light upon the light guide.

7. The endoscopic system according to claim 2, wherein the light emitting element emits the light, when the light source of the illuminating device is turned off or interrupted.

8. The endoscopic system according to claim 1, wherein the operation section includes an operation section main body through which the light guide is inserted and which is disposed in the proximal end of the insertion section; and a universal cord extended from the operation section main body and having a cord end portion connected to the illuminating device and provided with an incidence end portion of the light guide,
the light guide includes at least two incidence end faces in the cord end portion, and
the light emitting element is disposed beside the light source of the illuminating device, and detachably disposed in a position which faces at least one of the incidence end faces.

9. The endoscopic system according to claim 8, wherein the light emitting element emits the light in a state in which the light is emitted from the light source of the illuminating device.

10. The endoscopic system according to claim 9, wherein an RGB filter which feeds R-light, G-light and B-light to the light guide in order by rotation is disposed between the light source and the incidence end face of the light guide, and
the light emitting element emits the light while matching with an incidence timing of the R-light, the G-light and the B-light upon the light guide.

11. The endoscopic system according to claim 8, wherein the light emitting element emits the light, when the light source of the illuminating device is turned off or interrupted.

12. The endoscopic system according to claim 1, wherein the light emitting element is disposed in the emission end face of the light guide.

13. The endoscopic system according to claim 12, wherein the light emitting element is buried in the emission end face of the light guide.

14. The endoscopic system according to claim 12, wherein the light emitting element emits the light in a state in which the light is emitted from the light source of the illuminating device.

15. The endoscopic system according to claim 14, wherein an RGB filter which feeds R-light, G-light and B-light to the light guide in order by rotation is disposed between the light source and the incidence end face of the light guide, and
the light emitting element emits the light while matching with an incidence timing of the R-light, the G-light and the B-light upon the light guide.

16. The endoscopic system according to claim 12, wherein the light emitting element emits the light, when the light source of the illuminating device is turned off or interrupted.

17. The endoscopic system according to claim 1, wherein the light emitting element emits the light in a state in which the light is emitted from the light source of the illuminating device.

18. The endoscopic system according to claim 17, wherein an RGB filter which feeds R-light, G-light and B-light to the light guide in order by rotation is disposed between the light source and the incidence end face of the light guide, and
the light emitting element emits the light while matching with an incidence timing of the R-light, the G-light and the B-light upon the light guide.

19. The endoscopic system according to claim 1, wherein the light emitting element emits the light, when the light source of the illuminating device is turned off or interrupted.

20. An endoscopic system comprising:
an endoscope including:
an elongated insertion section having a distal end and a proximal end;
an operation section disposed in the proximal end of the insertion section; and
a light guide inserted through the operation section and the insertion section, having an incidence end face in the operation section, and having an emission end face in the distal end of the insertion section; and
an illuminating device optically connected to the incidence end face of the light guide disposed in the operation section, and having a light source which feeds light to the incidence end face,
wherein the insertion section is provided with, on the distal end of the insertion section, a light emitting element which emits light in parallel with the light-source light emitted from the emission end face.

21. The endoscopic system according to claim 20, wherein the light emitting element is integrally disposed in the emission end face of the light guide, and the emission end face of the light emitting element is the same plane as that of the emission end face of the light guide.

22. The endoscopic system according to claim 20, wherein the emission end face of the light emitting element is disposed beside the emission end face of the light guide.

23. The endoscopic system according to claim 20, wherein the light emitting element emits the light in a state in which the light is emitted from the light source of the illuminating device.

24. The endoscopic system according to claim 23, wherein an RGB filter which feeds R-light, G-light and B-light to the light guide in order by rotation is disposed between the light source and the incidence end face of the light guide, and
the light emitting element emits the light while matching with an incidence timing of the R-light, the G-light and the B-light upon the light guide.

25. The endoscopic system according to claim 20, wherein the light emitting element emits the light, when the light source of the illuminating device is turned off or interrupted.
